(19) United States
(12) Patent Application Publication
(10) Pub. No.: US 2020/0155078 A1
(43) Pub. Date: May 21, 2020

(54) HEALTH MONITORING USING ARTIFICIAL INTELLIGENCE BASED ON SENSOR DATA

(71) Applicant: INTERNATIONAL BUSINESS MACHINES CORPORATION, Armonk, NY (US)

(72) Inventors: Li Jun Mei, Beijing (CN); Qi Cheng Li, Beijing (CN); Xue Han, Beijing (CN); Lian Xue Hu, Beijing (CN)

(21) Appl. No.: 16/193,118

(22) Filed: Nov. 16, 2018

(51) Int. Cl. A61B 5/00; G16H 50/30; G16H 10/60; G06N 3/08; A61B 5/11

(52) U.S. Cl. CPC A61B 5/7267; G16H 50/30; G16H 10/60; G06N 3/08; A61B 5/7275; A61B 2562/0219; A61B 5/7203; A61B 5/1114; A61B 5/6824; A61B 5/6828; A61B 5/746

(57) ABSTRACT

A computer-implemented method for health monitoring using artificial intelligence based on sensor data includes collecting sensor data from one or more wearable devices affixable to a user, each wearable device including one or more sensors, predicting a risk of premonitory symptoms based on the sensor data by using a neural network model, and transmitting an alert to one or more entities associated with the user based on the predicted risk.

### BACKGROUND

### Technical Field

The present invention generally relates to artificial intelligence and machine learning, and more particularly to health monitoring using artificial intelligence based on sensor data.

### Description of the Related Art

Premonitory or prodromal symptoms refer to early signs or symptoms of a disease or illness that can indicate the onset of the disease. For example, people suffering from hypoglycemia can have premonitory symptoms including a tremble, which can occur before more serious symptoms. Therefore, identifying the early onset of a disease based on premonitory symptoms can improve the prognosis of a person with the disease.

### SUMMARY

In accordance with an embodiment of the present invention, a system for health monitoring using artificial intelligence based on sensor data is provided. The system includes one or more wearable devices affixable to a user. Each wearable device includes one or more sensors. The system further includes a memory device for storing program code and at least one processor operatively coupled to the memory device. The at least one processor is configured to execute program code stored on the memory device to collect sensor data from the one or more wearable devices, predict a risk of premonitory symptoms based on the sensor data by using a neural network model, and transmit an alert to one or more entities associated with the user based on the predicted risk.

In accordance with another embodiment of the present invention, a computer-implemented method for health monitoring based on sensor data is provided. The method includes collecting sensor data from one or more wearable devices affixable to a user, each wearable device including one or more sensors, predicting a risk of premonitory symptoms based on the sensor data by using a neural network model, and transmitting an alert to one or more entities associated with the user based on the predicted risk.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 is a block diagram of a processing system, in accordance with an embodiment of the present invention;

FIG. 2 is a block diagram of an illustrative cloud computing environment having one or more cloud computing nodes with which local computing devices used by cloud consumers communicate, in accordance with an embodiment of the present invention;

FIG. 3 is a block diagram of a set of functional abstraction layers provided by a cloud computing environment, in accordance with an embodiment of the present invention;

FIG. 4 is a block/flow diagram of a system/method for training a neural network to implement health monitoring based on sensor data, in accordance with an embodiment of the present invention;

FIG. 5 is a block/flow diagram of a system/method for using a neural network to implement health monitoring based on sensor data, in accordance with an embodiment of the present invention;

FIG. 6 is a diagram of sensor data collection and pattern recognition, in accordance with an embodiment of the present invention;

FIG. 7 is a block/flow diagram of sensor data transformation using neural networks, in accordance with an embodiment of the present invention; and

FIG. 8 is a diagram of a wearable device sensor system, in accordance with an embodiment of the present invention.

### DETAILED DESCRIPTION

A wearable device can include one or more sensors for measuring data associated with a user or object. Examples of types of sensors that can be implemented in wearable devices include, but are not limited to, accelerometers, gyroscopes, altimeters, and optical heart rate monitors.

Accelerometers can be used to measure acceleration, which can be used to measure speed of motion and distance traveled. In wearable devices, accelerometers can be used, possibly with other sensors, to determine, e.g., the number of steps and sleep quality of the user. For example, by measuring how fast a user moves, an accelerometer can be used to determine whether the user is walking, shaking a body part, etc., and by measuring how long a user is idle, an accelerometer can be used to determine whether the user is asleep.

Gyroscopes can be used to measure and/or maintain orientation and angular velocity, and can improve the accuracy of motion and activity tracking. In wearable devices, gyroscopes can be used to, e.g., distinguish whether a user is running or cycling.

Altimeters can be used to measure the altitude of an object above a fixed level (e.g., using atmospheric pressure). In wearable devices, altimeter data can be used to, e.g., determine a total flight of stairs that a user has climbed. This can be used to more accurately determine calorie consumption.

Optical heart rate monitors can be used to detect heart rate by shining a light (e.g., red or green light) against the skin to measure blood pumping. Optical heart rate monitors are generally implemented on wearable devices designed to be worn on the wrist.

The sensors described above are unable to directly detect premonitory symptoms in an effective manner. To address at least these drawbacks, the embodiments described herein can implement health monitoring using artificial intelligence based on sensor data. For example, the embodiments described herein can implement preemptive disease monitoring. More specifically, a neural network (e.g., convolutional neural network (CNN)) can be trained based on sensor data collected from one or more wearable devices including one or more sensors worn by a user. More specifically, the collected sensor data can be transformed into a graph, and the neural network can be trained to read the graph to understand the features of premonitory symptoms of diseases. During runtime, current sensor data can be obtained from one or more wearable devices including one or more sensors, and provided as input into a trained neural network model. The trained neural network model can be used to predict a risk of premonitory symptoms based on the current sensor data. If there is a risk of premonitory symptoms, an alert can be transmitted to one or more entities (e.g., the user, one or more doctors of the user, or any other potentially interested party) informing the one or more entities of the risk.

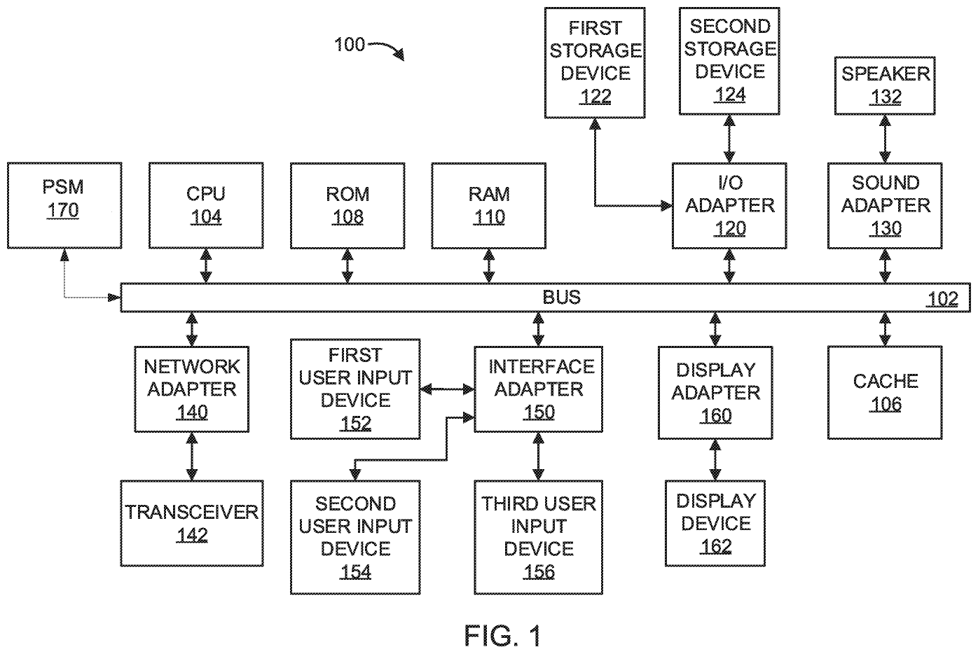

Referring now to the drawings in which like numerals represent the same or similar elements and initially to FIG. 1, an exemplary processing system 100 to which the present invention may be applied is shown in accordance with one embodiment. The processing system 100 includes at least one processor (CPU) 104 operatively coupled to other components via a system bus 102. A cache 106, a Read Only Memory (ROM) 108, a Random Access Memory (RAM) 110, an input/output (I/O) adapter 120, a sound adapter 130, a network adapter 140, a user interface adapter 150, and a display adapter 160, are operatively coupled to the system bus 102.

A first storage device 122 and a second storage device 124 are operatively coupled to system bus 102 by the I/O adapter 120. The storage devices 122 and 124 can be any of a disk storage device (e.g., a magnetic or optical disk storage device), a solid state magnetic device, and so forth. The storage devices 122 and 124 can be the same type of storage device or different types of storage devices.

A speaker 132 is operatively coupled to system bus 102 by the sound adapter 130. A transceiver 142 is operatively coupled to system bus 102 by network adapter 140. A display device 162 is operatively coupled to system bus 102 by display adapter 160.

A first user input device 152, a second user input device 154, and a third user input device 156 are operatively coupled to system bus 102 by user interface adapter 150. The user input devices 152, 154, and 156 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 152, 154, and 156 can be the same type of user input device or different types of user input devices. The user input devices 152, 154, and 156 are used to input and output information to and from system 100.

Premonitory symptom monitoring (PSM) component 170 may be operatively coupled to system bus 102. PSM component 170 is configured to perform one or more of the operations described below. PSM component 170 can be implemented as a standalone special purpose hardware device, or may be implemented as software stored on a storage device. In the embodiment in which PSM component 170 is software-implemented, although the anomaly detector is shown as a separate component of the computer system 100, PSM component 170 can be stored on, e.g., the first storage device 122 and/or the second storage device 124. Alternatively, PSM component 170 can be stored on a separate storage device (not shown).

Of course, the processing system 100 may also include other elements (not shown), as readily contemplated by one of skill in the art, as well as omit certain elements. For example, various other input devices and/or output devices can be included in processing system 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 100 are readily contemplated by one of ordinary skill in the art given the teachings of the present invention provided herein.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

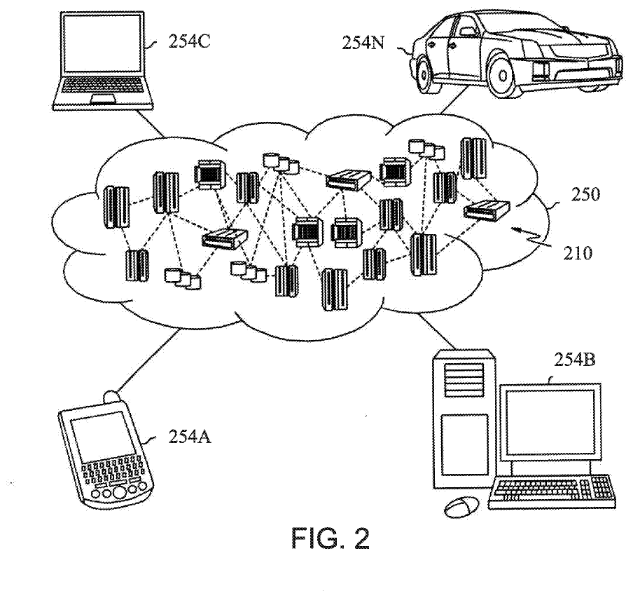

Referring now to FIG. 2, illustrative cloud computing environment 250 is depicted. As shown, cloud computing environment 250 includes one or more cloud computing nodes 210 with which local computing devices used by cloud consumers, such as, for example, personal digital assistant (PDA) or cellular telephone 254A, desktop computer 254B, laptop computer 254C, and/or automobile computer system 254N may communicate. Nodes 210 may communicate with one another. They may be grouped (not shown) physically or virtually, in one or more networks, such as Private, Community, Public, or Hybrid clouds as described hereinabove, or a combination thereof. This allows cloud computing environment 150 to offer infrastructure, platforms and/or software as services for which a cloud consumer does not need to maintain resources on a local computing device. It is understood that the types of computing devices 254A-N shown in FIG. 2 are intended to be illustrative only and that computing nodes 210 and cloud computing environment 250 can communicate with any type of computerized device over any type of network and/or network addressable connection (e.g., using a web browser).

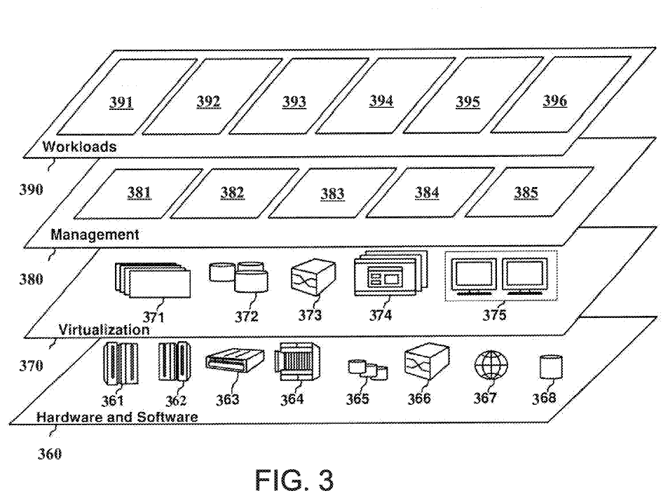

Referring now to FIG. 3, a set of functional abstraction layers provided by cloud computing environment 250 (FIG. 2) is shown. It should be understood in advance that the components, layers, and functions shown in FIG. 3 are intended to be illustrative only and embodiments of the invention are not limited thereto. As depicted, the following layers and corresponding functions are provided:

Hardware and software layer 360 includes hardware and software components. Examples of hardware components include: mainframes 361; RISC (Reduced Instruction Set Computer) architecture based servers 362; servers 363; blade servers 364; storage devices 365; and networks and networking components 366. In some embodiments, software components include network application server software 367 and database software 368.

Virtualization layer 370 provides an abstraction layer from which the following examples of virtual entities may be provided: virtual servers 371; virtual storage 372; virtual networks 373, including virtual private networks; virtual applications and operating systems 374; and virtual clients 375.

In one example, management layer 380 may provide the functions described below. Resource provisioning 381 provides dynamic procurement of computing resources and other resources that are utilized to perform tasks within the cloud computing environment. Metering and Pricing 382 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 383 provides access to the cloud computing environment for consumers and system administrators. Service level management 384 provides cloud computing resource allocation and management such that required service levels are met. Service Level Agreement (SLA) planning and fulfillment 385 provide pre-arrangement for, and procurement of, cloud computing resources for which a future requirement is anticipated in accordance with an SLA.

Workloads layer 390 provides examples of functionality for which the cloud computing environment may be utilized. Examples of workloads and functions which may be provided from this layer include: mapping and navigation 391; software development and lifecycle management 392; virtual classroom education delivery 393; data analytics processing 394; transaction processing 395; and health monitoring 396.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

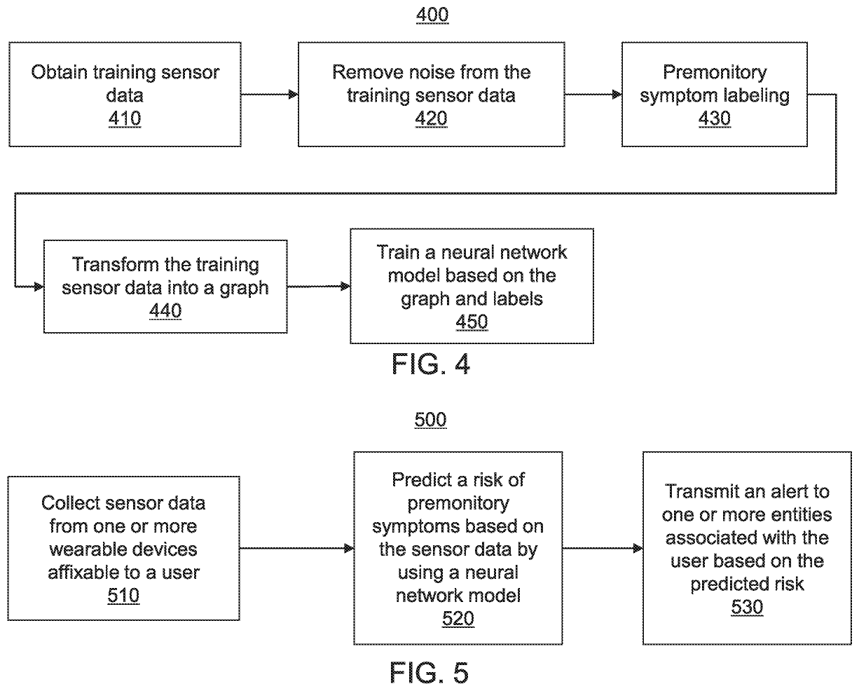

With reference to FIG. 4, a block/flow diagram is provided illustrating a system/method 400 for training a neural network to implement health monitoring based on sensor data.

At block 410, data including training sensor data is obtained. The training sensor data can include data corresponding to body movement traces collected from one or more wearable devices each including one or more sensors. In one embodiment, the one or more sensors can include one or more gyroscopes for monitoring the body movement traces. For example, a wearable device including at least one gyroscope can be worn on each arm and leg of a user.

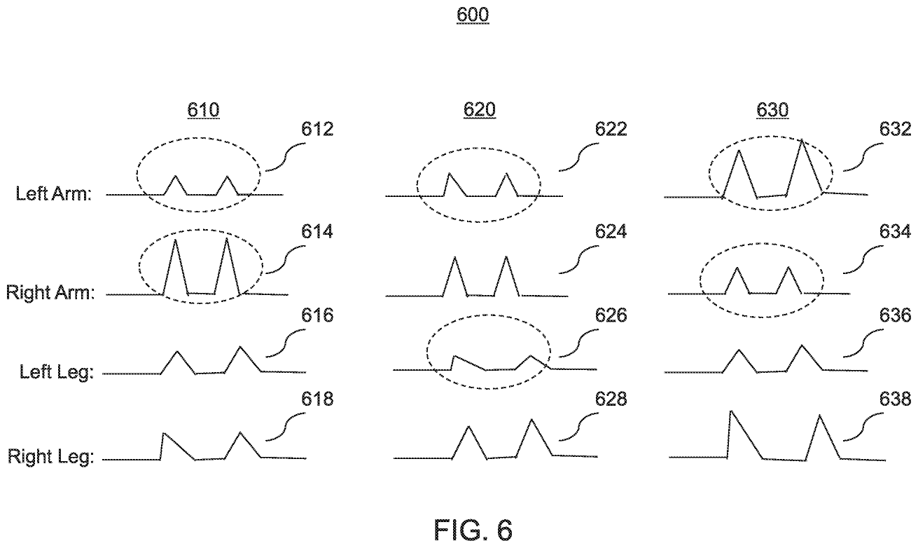

In one embodiment, the data obtained at block 410 can further include human labels that provide a source of training data for the neural network. The human labels can be obtained from users, and correspond to instances where a user does not feel well. For example, when a user does not feel well, the user can record discomfort and/or symptoms. Further details regarding sensor data collection will be described below with reference to FIG. 6.

At block 420, the training sensor data is processed. In one embodiment, processing the training sensor data can include removing noise from the training sensor data. For example, processing the training sensor data can include performing cross-validation to remove noise from the training sensor data.

At block 430, premonitory symptom labeling is performed. In one embodiment, performing premonitory symptom labeling can include automatically generating labels for premonitory symptoms of different diseases based at least in part on the processed training sensor data. Other trackable environmental data can also be used with the processed training sensor data to automatically generate the labels. Examples of other trackable environmental data can include, but are not limited to, surveillance video data, body sensor data, hospital records (e.g., call records and nursing records), etc. For example, surveillance video or body sensor data can be analyzed to detect, e.g., fainting, passing out, and trembling, while disease symptoms can be extracted from call records and/or nursing records. The premonitory symptom labeling performed at block 430 can be performed to supplement any human labels that are obtained at block 410, or can be performed without obtaining any prior human labels.

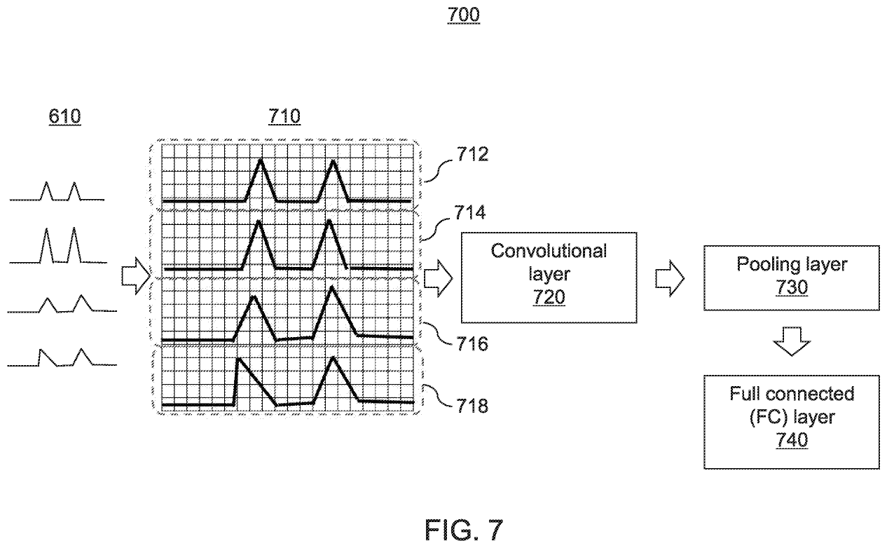

At block 440, the training sensor data is transformed into a graph. In one embodiment, the graph includes a two-dimensional (2D) graph. At block 450, a neural network model is trained based on the graph. In one embodiment, the neural network model includes a convolutional neural network (CNN) model. Human labels obtained at block 410 and/or labels generated at block 430 can be used to train the neural network model. Further details regarding blocks 440 and 450 will be described below with reference to FIG. 7.

With reference to FIG. 5, a block diagram is provided illustrating a system/method 500 for using a neural network to implement health monitoring based on sensor data.

At block 510, sensor data is collected from one or more wearable devices affixable to a user. For example, the one or more wearable devices can be worn on at least one appendage of the user. The sensor data can be collected in real-time or near real-time. Each wearable device can include one or more sensors configured to collect data corresponding to body movement traces. In one embodiment, the one or more sensors can include one or more gyroscopes for monitoring the body movement traces. For example, a wearable device including at least one gyroscope can be worn on each arm and leg of a user. Further details regarding sensor data collection will be described below with reference to FIG. 6.

At block 520, a neural network model is used to predict a risk of premonitory symptoms based on the sensor data. In one embodiment, the neural network model includes a convolutional neural network (CNN) model. For example, the neural network model can be a neural network model trained in accordance with the system/method described above with reference to FIG. 4. Further details regarding block 520 will be described below with reference to FIG. 7.

At block 530, if there is a risk of premonitory symptoms, an alert is transmitted to one or more entities associated with the user. The one or more entities can include the user, one or more doctors associated with the user, etc. The alert can be transmitted to one or more electronic devices associated with the one or more entities. For example, the alert can be transmitted to at least one of the one or more wearable devices worn on the user. As another example, the alert can be transmitted as an electronic message delivered to the user (e.g., e-mail or text message).

With reference to FIG. 6, a diagram 600 is provided illustrating sensor data collection and pattern recognition. As shown, the diagram 600 includes a plurality of sensor data collections, including sensor data collection 610, sensor data collection 620 and sensor data collection 630. The sensor data collections 610-630 can include sensor data collected from one or more sensors such as, e.g., at least one accelerometer, at least one gyroscope, at least one altimeter, at least one optical heart rate monitor, and combinations thereof.

In this illustrative example, it is assumed that sensor data in each sensor data collection 610-630 is obtained from four sensors, with each sensor being embodied within a wearable device on one of a left arm, right arm, left leg and right leg of a user. In one embodiment, each of the four sensors includes a gyroscope embodied within a wearable device on the left arm, right arm, left leg and right leg of the user. More specifically, as shown, sensor data collection 610 includes left arm sensor data 612, right arm sensor data 614, left leg sensor data 616 and right leg sensor data 618. Sensor data collection 620 includes left arm sensor data 622, right arm sensor data 624, left leg sensor data 626 and right leg sensor data 628. Sensor data collection 630 includes left arm sensor data 632, right arm sensor data 634, left leg sensor data 636 and right leg sensor data 638.

As further shown, in sensor data collection 610, portions of left arm sensor data 612 and right arm sensor data 614 are circled, in sensor data collection 620, portions of left arm sensor data 622 and left leg sensor data 626 are circled, and in sensor data collection 630, portions of left arm sensor data 632 and right arm sensor data 634 are circled. The combined circled portions of the sensor data (e.g., sensor data 612 and 614, 622 and 626, and 632 and 634) can be used to predict the premonitory symptoms for the disease. For example, if circled portions 612 and 614 appear at the same time for the left arm and right arm, this can imply a premonitory symptom for a certain disease. The circled portions can be determined by the trained neural network.

With reference to FIG. 7, a block/flow diagram is provided illustrating an exemplary system/method 700 for performing sensor data transformation using neural networks. As shown, a sensor data collection, which in this illustrative example is sensor data collection 610 described above with reference to FIG. 6, is transformed into a graph 710 corresponding to an image of the sensor data. The graph 710 includes a plurality of regions 712-718, which each region corresponding to sensor data 612-618 within the sensor data collection 610, respectively.

The graph 710 is input into a neural network to predict a risk of premonitory symptoms based on the graph 710. In this illustrative example, the neural network is a convolutional neural network (CNN). However, other types of neural networks can be implemented in accordance with the embodiments described herein.

As shown, the graph 710 is input into a convolutional layer 720. The convolutional layer 720 applies a convolution operation to the graph 710 using one or more filters to generate an activation or feature map. The convolutional layer 720 can use any number of filters in accordance with the embodiments described herein to generate the feature map.

The output of the convolutional layer 720 is fed into a pooling layer 730. The pooling layer 730 uses a filter to down-sample the output of the convolutional layer 720. In one embodiment, the pooling layer 730 implements max pooling. Max pooling applies a filter to the output of the convolutional layer 720 and outputs the maximum number in every sub-region covered by the filter. However, other pooling functions (e.g., average pooling and/or L2-norm pooling) can also be used in accordance with the embodiments described herein. The pooling layer 730 can reduce computational costs of implementing the neural network for preemptive disease monitoring, and can reduce the effects of overfitting.

The output of the pooling layer 730 is fed into a fully connected (FC) layer 740. The FC layer 740 can determine which features most correlate to a class (e.g., disease) by looking at which high level features most strongly correlate to the class. For example, the FC layer 740 can output an N dimensional vector, where N is the number of classes (e.g., diseases), and each number in the vector represents a probability of the class (e.g., softmax).

Other layers can be included in the neural network. For example, multiple convolutional and pooling layers can be used. Moreover, one or more layers with non-linear activation functions, such as, e.g., Rectified Linear Unit (ReLU), can be placed within the neural network.

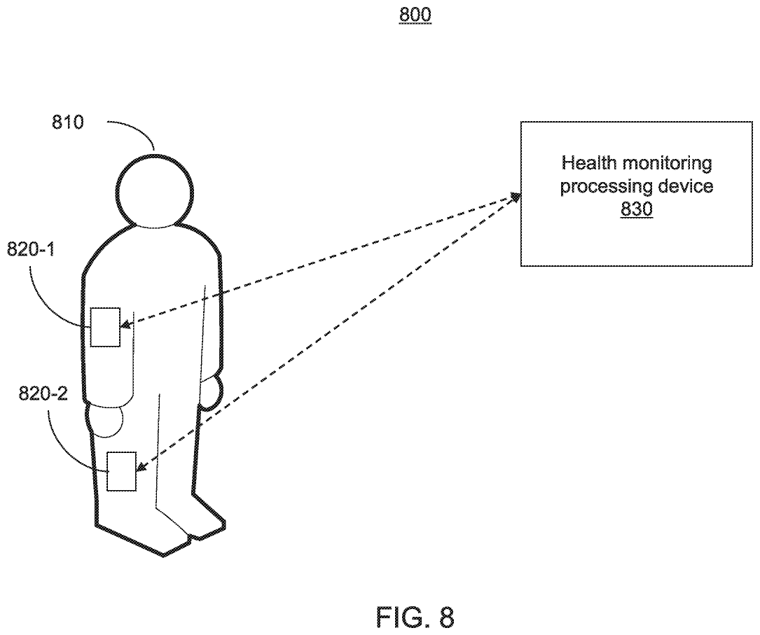

With reference to FIG. 8, a diagram is provided illustrating a wearable device sensor system for health monitoring 800. As shown, a user 810 is affixed with a wearable device 820-1 on the right arm and a wearable device 820-2 on the right leg. Each wearable device 820-1 and 820-2 can include one or more sensors for measuring data associated with a user or object. Examples of types of sensors that can be implemented in the wearable devices 820-1 and 820-2 include, but are not limited to, accelerometers, gyroscopes, altimeters, and optical heart rate monitors. In an illustrative embodiment, wearable devices including respective gyroscopes can be affixed to the arm and legs of the user 810.

However, such an embodiment should not be considered limiting. For example, the user 810 can be affixed with any number of wearable devices on any number of appendages in accordance with the embodiments described herein.

The wearable devices 820-1 and 820-2 are configured to communicate with a health monitoring processing device 830 via a network. For example, the health monitoring processing device can include, e.g., a server. The health monitoring processing device 830 is configured to receive or collect sensor data from the wearable devices 820-1 and 820-2, and perform health monitoring using artificial intelligence based on the sensor data. For example, the health monitoring processing device 830 can predict a risk of premonitory symptoms based on the sensor data by using a neural network model, and transmit an alert to one or more entities associated with the user based on the predicted risk. Further details regarding the system 800 are described above with reference to FIGS. 1-7.

Having described preferred embodiments of systems and methods of health monitoring using artificial intelligence based on sensor data (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed:

1. A system for health monitoring using artificial intelligence based on sensor data, comprising:
one or more wearable devices affixable to a user, each wearable device including one or more sensors;
a memory device for storing program code; and
at least one processor operatively coupled to the memory device and configured to execute program code stored on the memory device to:
collect sensor data from the one or more wearable devices;
predict a risk of premonitory symptoms based on the sensor data by using a neural network model; and
transmit an alert to one or more entities associated with the user based on the predicted risk.

2. The system of claim 1, wherein the at least one sensor includes at least one gyroscope.

3. The system of claim 1, wherein the one or more wearable devices are configured to worn on at least one appendage of the user.

4. The system of claim 1, wherein the at least one processor is configured to transmit the alert to the user, one or more persons associated with the user, and combinations thereof.

5. The system of claim 1, wherein the at least one processor is further configured to train the neural network model based on training sensor data.

6. The system of claim 5, wherein the at least one processor is further configured to train the neural network model by:
obtaining the training sensor data;
transforming the training sensor data into a graph; and
training the neural network model based on the graph and labels.

7. The system of claim 6, wherein the at least one processor is further configured to train the neural network model by removing noise from the training sensor data.

8. A computer-implemented method for health monitoring using artificial intelligence based on sensor data, comprising:
collecting sensor data from one or more wearable devices affixable to a user, each wearable device including one or more sensors;
predicting a risk of premonitory symptoms based on the sensor data by using a neural network model; and
transmitting an alert to one or more entities associated with the user based on the predicted risk.

9. The method of claim 8, wherein the at least one sensor includes at least one gyroscope.

10. The method of claim 8, wherein the one or more wearable devices are configured to worn on at least one appendage of the user.

11. The method of claim 8, wherein transmitting the alert further comprises transmitting transmit the alert to the user, one or more persons associated with the user, and combinations thereof.

12. The method of claim 8, further comprising training the neural network model based on training sensor data.

13. The method of claim 12, wherein training the neural network model further includes:
obtaining the training sensor data;
transforming the training sensor data into a graph; and
training the neural network model based on the graph and labels.

14. The method of claim 13, wherein training the neural network model further includes removing noise from the training sensor data.

15. A computer program product comprising a non-transitory computer readable storage medium having program instructions embodied therewith, the program instructions executable by a computer to cause the computer to perform a method for health monitoring using artificial intelligence based on sensor data, the method performed by the computer comprising:
collecting sensor data from one or more wearable devices affixable to a user, each wearable device including one or more sensors;
predicting a risk of premonitory symptoms based on the sensor data by using a neural network model; and
transmitting an alert to one or more entities associated with the user based on the predicted risk.

16. The computer program product of claim 15, wherein the at least one sensor includes at least one gyroscope.

17. The computer program product of claim 15, wherein the one or more wearable devices are configured to worn on at least one appendage of the user.

18. The computer program product of claim 15, wherein transmitting the alert further comprises transmitting transmit the alert to the user, one or more persons associated with the user, and combinations thereof.

19. The computer program product of claim 15, wherein the method further comprises training the neural network model based on training sensor data, including:
obtaining the training sensor data;
transforming the training sensor data into a graph; and
training the neural network model based on the graph and labels.

20. The computer program product of claim 19, wherein training the neural network model further includes removing noise from the training sensor data.
